(19) United States
(12) Patent Application Publication
(10) Pub. No.: US 2018/0358119 A1
(43) Pub. Date: Dec. 13, 2018

(54) METHOD AND SYSTEM FOR CONTINUOUS MONITORING OF HEALTH PARAMETERS DURING EXERCISE

(71) Applicant: FOURTH FRONTIER TECHNOLOGIES, Pvt. Ltd., Bangalore (IN)

(72) Inventors: MANAV BHUSHAN, BANGALORE (IN); SANDEEP SIBAL, BANGALORE (IN); Ravali Amba, BANGALORE (IN)

(73) Assignee: FOURTH FRONTIER TECHNOLOGIES, Pvt. Ltd., Bangalore (IN)

(21) Appl. No.: 16/035,706

(22) Filed: Jul. 16, 2018

Related U.S. Application Data
(63) Continuation-in-part of application No. 15/465,069, filed on Mar. 21, 2017.

(30) Foreign Application Priority Data
Jun. 3, 2016 (IN) 201641019151
Jun. 3, 2016 (IN) 201641019154
Jun. 3, 2016 (IN) 201641019157
Jun. 3, 2016 (IN) 201641019197
Jan. 13, 2018 (IN) 201841001552

(51) Int. Cl. G16H 20/30; G06F 1/16; G16H 50/20; A61B 5/0205; A61B 5/00; A63B 24/00

(52) U.S. Cl. CPC G16H 20/30; G06F 1/163; G16H 50/20; A61B 5/02055; A61B 5/7405; A61B 5/742; A61B 5/01; A61B 5/746; A61B 5/681; A61B 5/486; A61B 5/6898; A63B 24/0062; A61B 5/7455

(57) ABSTRACT

The various embodiments of the present invention provide a system and method for a fully mobile, non-invasive, continuous system for monitoring the cardiovascular and musculoskeletal health of an individual during exercise. The system includes one or more wearable devices affixed on the User with a chest strap or adhesive sticker, coupled with an application running on a computing device (smartphone/smartwatch), which performs various computations on the wearable device, or a smart phone/smart watch, or the cloud, and provides the user or the concerned personnel with various insights about the general health of the user. The exercise health monitoring system further enables the Users to get real time alerts during exercise or running, by way of vibrations or audio messages or notifications on the gateway device when they have an event of arrhythmia or cardiac fatigue, or they cross prescribed ranges of one or more of the following parameters: PEP, LVET, CO, HR, Shock, Braking Force, Sway.

### BACKGROUND

The present invention is generally related to health monitoring devices. The present invention is particularly related to a device and system for monitoring cardiovascular and musculoskeletal health and analysing healthcare data. The present invention is more particularly related to cardiovascular and musculoskeletal health monitoring, and the analysis and provision of real-time alerts using wearable devices.

### PRIOR ART

Patent application US2015/0099945A1 describes an activity monitoring device (AMD) that measures Heart Rate, running style, cadence, biking posture, etc.

Patent application US2015/0099945A1 (Wahoo) describes an activity monitoring device (AMD) that measures Heart Rate, running speed, ground contact time, vertical oscillation, cadence, biking posture, etc.

Patent application U.S. Pat. No. 9,699,859B1 (Moov) discloses an automated fitness coaching device, comprising light-emitting diodes (LEDs) and multiple sensors, giving guidance through audio messages delivered through the phone to improve running styles.

Patent application US2013/0178958A1 (Garmin) discloses a system comprising of an inertial sensor coupled to the User's torso, measuring speed, cadence, time energy cost, distance energy cost and acceleration energy cost.

The U.S. Pat. No. 8,630,867 discloses a system and method for remote diagnosis using a wearable device. The patent also discloses a system and method for a user to communicate with a number of doctors/specialists through the wearable device which is paired with a computing device such as a Smartphone.

The U.S. Pat. No. 8,107,920 discloses a wearable health monitoring system. According to this patent, one or more concerned personnel are alerted when a user's condition is critical and is below a set threshold. The patent also teaches measuring parameters such as heart rate, respiration rate, and the like using sensors available on the wearable device.

The patent application US20160210434 discusses a system and method for storing medical records using a wearable device. The patent application discusses securing an appointment with a concerned personnel/doctor through the wearable device. In addition, the patent application also discloses transmitting the required diagnostic reports to the caregiver.

The fitness industry suffers from some acute problems, including but not limited to increased risk of injuries to the knees and spine, as well as over-exertion resulting in cases of myocardial infarction.

To effectively circumvent this problem of over-exertion and injuries, a system that can effectively monitor cardiovascular health parameters, and musculo-skeletal health, would be very useful. Further, a system that allows a User to monitor these parameters during exercise, without the need of an accompanying gateway device such as a phone or watch would be more effective in preventing injuries and adverse effects on the cardiovascular system.

### SUMMARY

The various embodiments of the present invention provide a system and method for continuous health monitoring of the User during exercise. The system includes a wearable device that is coupled with a chest strap, which allows the device to be attached to the body of the User. The wearable device includes a plurality of electrodes, an electronic circuitry to measure electric potentials for one or more channels, and/or a circuitry for measuring electrical impedance on the skin using electrodes, and/or one or more accelerometers, and/or a reflectance-based Photoplethysmograph (PPG) sensor module. The wearable device is designed to measure the electrocardiogram (ECG) and/or heart rate and/or respiration cycles and/or blood oxygenation and/or the seismocardiography (SCG) and/or body movements and/or blood pressure (BP). The wearable device may also include a blood glucose sensor, and/or a sensor to measure levels of Haemoglobin (Hb) and other blood gases (such as Carbon Dioxide) of the user.

The various embodiments of the present invention provide a system wherein a wearable device capable of monitoring various physiological signals also has a processor capable of recording data to a memory chip on the device, and computing different metrics, without the need for any external device. The device is further capable of sending alerts to the User by way of LEDs situated on the device, and/or an electronic display, and/or a vibration motor, and/or an audio speaker located on the device.

The various embodiments of the present invention provide a system that includes a wearable device located on the torso of the User, which includes a wireless communication module (Bluetooth and/or WiFi and/or NFC) capable of communicating raw data and various metrics computed on the wearable device to a gateway device such as a smartphone or a smartwatch.

The various embodiments of the present invention provide systems and methods for monitoring and analysing bio-signals measured by one or more wearable devices, and alerting the user in real-time, during exercise when certain conditions are detected.

These and other aspects of the embodiments herein will be better appreciated and understood when considered in conjunction with the following description and the accompanying drawings. It should be understood, however, that the following descriptions, while indicating the preferred embodiments and numerous specific details thereof, are given by way of an illustration and not of a limitation. Many changes and modifications may be made within the scope of the embodiments herein without departing from the spirit thereof, and the embodiments herein include all such modifications.

The other objects, features, and advantages will occur to those skilled in the art from the following description of the preferred embodiment and the accompanying drawings.

### BRIEF DESCRIPTION OF THE DRAWINGS

FIG. 1 shows a schematic layout of the Wearable Device (115) and shows a plurality of sensors (101, 102, 103, 104), that record data, and send the data to a microcontroller or microprocessor or another computing device (105) on the Wearable.

FIG. 2 shows a picture of the internal components of the Wearable (115), in the actual configuration, as used in the product, according to various embodiments.

FIG. 3 shows a process flowchart for how the Wearable (115) and accompanying application function, in various embodiments.

FIG. 4 shows the process flowchart for detection of arrhythmia on the wearable device, in a mobile setting.

FIG. 5 shows the wearable device in one particular embodiment, where it can be worn with a double-sided sticker.

FIG. 6 shows the wearable device in one particular embodiment, stuck to the sternum of a User.

FIG. 7 shows the wearable device in one particular embodiment, where it can be affixed to the User's chest with the help of a chest-strap.

FIG. 8 shows various embodiments of the health monitoring system, comprising of the Wearable device (115), which sends the data collected from the User to the smart phone or gateway device (118). The smart phone (118) then sends the data to the web server (119), where the data is processed, stored and/or analyzed in greater detail. Thereafter, the system can send alerts to the User, caregiver and/or Doctor, in various embodiments.

FIG. 9 illustrates various embodiments of the process of monitoring a single patient.

FIG. 10 shows a block diagram for the collection of physiological data by multiple Wearables for multiple Users (125), in various embodiments.

FIG. 11 shows an example of the signals captured on the Wearable device (115), comprising of the ECG signal (132) and the SCG signal (133), which together denote several events of the cardiac cycle.

### DETAILED DESCRIPTION

In the following detailed description, a reference is made to the accompanying drawings that form a part hereof, and in which the specific embodiments that may be practiced is shown by way of illustration. These embodiments are described in sufficient detail to enable those skilled in the art to practice the embodiments and it is to be understood that the logical, mechanical and other changes may be made without departing from the scope of the embodiments. The following detailed description is therefore not to be taken in a limiting sense.

The various embodiments of the present invention provide a system and method for monitoring the health of a user continuously during exercise. The system comprises of a wearable device or devices which communicate wirelessly to a gateway device such as a smartphone/smartwatch/router. The wearable device(s) comprise of an electronic module or a component that is reusable and rechargeable (via any wire, such as a micro-USB/firewire/Pogo pins or wirelessly or both) or disposable and non-rechargeable, and is affixed to the body of the User with the help of a one-sided or two-sided adhesive, or with a strap, or a clip which ensures contact between the wearable device and the User's body (torso or foot).

In various embodiments, the Wearable device may be affixed to the body of the User with the help of a chest strap or a double-sided disposable sticker which covers a part or whole of the device, and also affixes to the skin around the wearable device, while ensuring direct contact between a part or whole of the wearable device, and some part of the User's torso or foot.

In various embodiments, the system includes a strap with two or more electrodes, and a device with two or more electrodes, which couples with the strap. The strap may include one or more energy harvesting chips that can harvest energy from the temperature difference between the User's skin and the ambient environment.

In various embodiments, the strap may also include various sensors including one or more of the following: a temperature sensor, a PPG sensor, an array of electrodes to measure ECG and/or skin conductance. This strap may couple with a device containing a wireless communication module, a microprocessor, and/or an accelerometer and other sensors.

In various embodiments, the Wearable device may be affixed to the skin of the User with the help of an adjustable or elastic band that fits around the User's torso, and which has a marking or cavity that holds the Wearable device in a particular desired location.

In various embodiments, the Wearable device may be placed in a cavity or specially designed appendage that is part of any article of clothing, such as a shirt or vest or harness that is in contact with some part of the Users chest. This piece of clothing would keep the Wearable device in a particular location on the User's chest.

In various embodiments, the Wearable device is fabricated upon a flexible printed circuit board (PCB), or on two or more hard PCBs connected with flexible PCBs (together making up a rigid-flex PCB), or on any combination of flexible or hard PCBs. According to an embodiment of the present invention, the width of the wearable device is 5-250 mm in length, 3-250 mm in width, and 1-250 mm in height, and at least part of the Wearable device is flexible, and adapts to almost any surface on the body including the forehead or abdomen or chest of the user.

In various embodiments, the Wearable device includes a vibration motor to alert the user under certain pre-defined circumstances. Alerts are sent when some abnormality is detected from the bio-signals being recorded—either as computed on a Multipoint Control Unit (MCU) itself in real-time, or as computed on the web server on the cloud and then communicated to the Wearable by way of Bluetooth of some other wireless communication protocol, or according to the findings of a doctor looking at the database on the web client communicated to the Wearable by way of Bluetooth of some other wireless communication protocol.

In various embodiments, the Wearable device includes one or more LEDs, visible through the casing, or placed on top of the casing, which communicates different information about the device status and functionality to the user, and/or a microprocessor or other processor to collect data from the multiple sensors, and perform different kinds of algorithms on the wearable device itself.

In various embodiments, the Wearable device includes an integrated circuit (IC) for wireless data communication, that enables it to connect and communicate and send and receive data from a smartphone/smartwatch or another gateway device. Further, the Wearable device may include a memory chip that allows it to store data for long periods of time, and then to communicate this saved data to other locations.

In various embodiments, the Wearable device contains an audio speaker that allows the User to hear certain alerts or audio commands. The Wearable device may also contain an audio recorder that allows the User to record or send audio instructions to the Wearable.

In various embodiments, the wearable device may be re-charged through a wired connection, such as a micro-USB connection/firewire/pogo pin, or through a wireless charger, and hence can be reused many times. Further, the Wearable device may have a casing which is waterproof, and may therefore be used in conditions where there are water and rain, or under water.

In various embodiments, the wearable device includes a gyroscope, which can calculate the exact orientation of the User while he/she is wearing the device on any part of the body. The gyroscope sends the data of the User's orientation to the computing device included in the wearable, and the User can be alerted when the orientation is falling outside a certain prescribed range, or when it changes more rapidly than a prescribed rate of change.

In various embodiments, the wearable device includes a magnetometer, which can calculate the orientation of the User with respect to the Earth's magnetic field, and provide a measurement for the direction in which the User is running/walking.

In various embodiments, the Wearable device includes one or more accelerometers capable of measuring acceleration within a range of 0.01 milliG-20 G, and hence capable of measuring steps, breathing, heartbeats, opening and closing of heartvalves (the aortic and mitral valves), rapid ejection and rapid filling, when placed at different locations on the body. The accelerometer(s) record Seismocardiography (SCG) when affixed to particular parts of the User's chest. The accelerometers may also include a tap-detection functionality, which allow the user to activate different kinds of processes with a single/double tap.

In various embodiments, the Wearable comprises of the following elements: two or more electrodes connected to a single analog front end (AFE) system, which may further transmit the signal to an analog-to-digital converter (ADC) and then on to an MCU. The Wearable device may also contain a digital PPG sensor and/or one or more accelerometers and/or a temperature sensor configured for measuring the skin temperature, at the location where the device is affixed to the body.

In various embodiments, the electrodes, AFE and MCU together measure and record electrical signals comprising of the Electrocardiogram (ECG) when stuck on the chest, or Electroencephalogram (EEG) when stuck on the forehead, or electromyogram (EMG) when stuck on a muscle, or a combination of all three signals.

In various embodiments, the Wearable device includes a reflective Photoplethysmograph (PPG) module attached to the underside of the device, and in direct visual contact with the skin on the chest/wrist/forehead or other location where the device adheres. The PPG module comprises of two or more light emitting diodes (LEDs), and one or more photodiodes, which measure the changes in the intensity of reflected light of one or more wavelengths. The PPG module may be capable of measuring blood oxygenation, and/or levels of Haemoglobin, and/or other blood gases, such as carbon dioxide (CO₂), and/or heart rate, and/or other measures derived from changes in blood flow. In various embodiments, the device uses the above-mentioned optical sensor, or a different sensor, emitting Electromagnetic waves at two or more wavelengths, to measure Blood Glucose levels.

In various embodiments, the Wearable device measures inhalation and exhalation cycles using an electrical impedance measured between two or more electrodes, and/or from the movements of the accelerometer(s), and/or from the signal measured on the PPG module, and/or from the variation in the magnitude of the R-peaks as measured on the ECG sensor.

In various embodiments, the Wearable device, when affixed vertically or horizontally on the sternum, or any other location on the chest, uses the SCG data collected from the accelerometer, to detect cardiac events including, but not limited to: Heart murmurs, Aortic valve opening (AO), Mitral valve opening (MO), Aortic valve closure (AC), Mitral valve closure (MC), Rapid Ejection (RE), Rapid Filling (RF), and Atrial Systole (AS), the peak after the AO event on the y-axis of the SCG (J-wave).

In various embodiments, the Wearable device uses the AFE sensor to record the ECG of the User during exercise, and calculates Heart Rate and/or Heart Rate Variability and/or ST-elevation from the ECG signal. The raw ECG data, and the derived parameters are stored on the flash chip in the wearable device, and/or communicated to the gateway device (smartphone or smartwatch) using a wireless communication protocol.

In various embodiments, the computing device contained in the wearable device estimates Potassium levels in blood using the following relationship:
[K⁺]=f(T-peak amplitude, Slope of ECG before and after T-peak, T-wave duration, R-peak amplitude, ST-elevation, Heart Rate, PEP)
Here [K⁺] is the concentration of Potassium in the blood, T-wave duration is the time duration (in ms) from the beginning till the end of the T-wave.

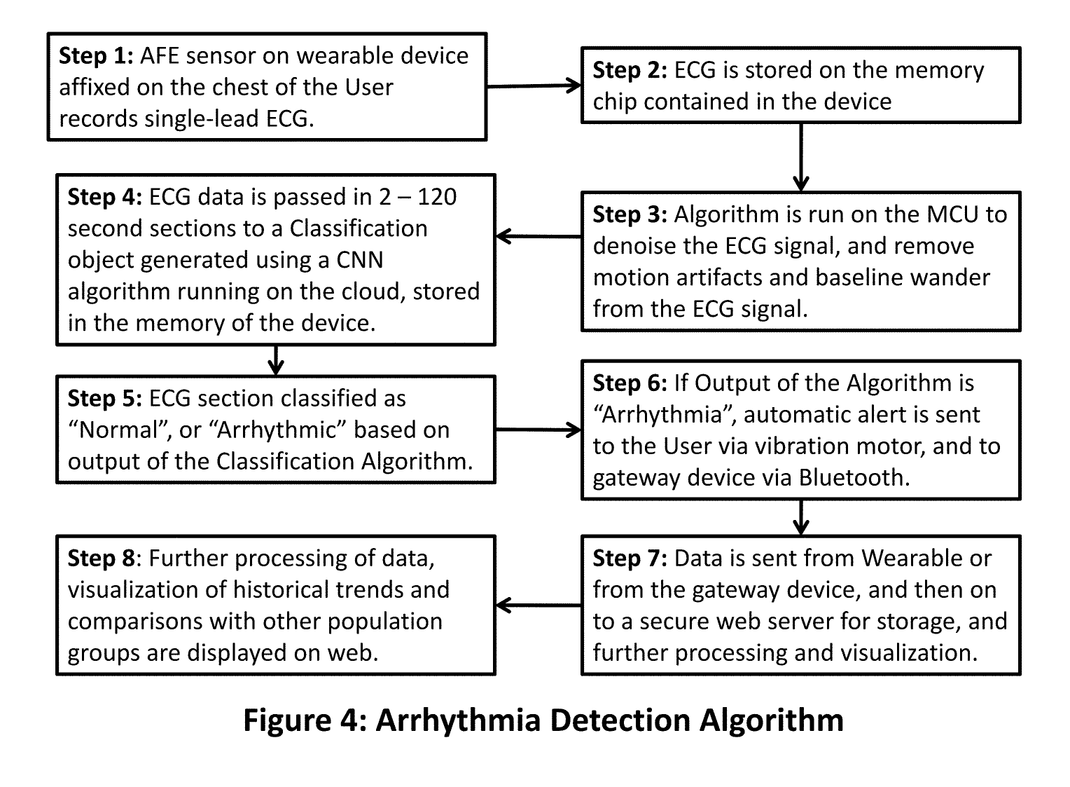

In various embodiments, the wearable device uses the ECG data to record arrhythmias in the User, by measuring the regularity of the heart rate variations on the basis of the RR-intervals recorded. The algorithm used to determine whether a particular beat is arrhythmic or not is described in FIG. 4.

In various embodiments, the wearable device records the ECG, and then uses a Convolutional Neural Network based algorithm for arrhythmia detection. The Network is trained on single-lead ECG data annotated by experts earlier, and the classification object is saved on the memory of the wearable device and/or the gateway device, and then the raw ECG data recorded on the wearable device is passed to the classification object, and the classification is stored on the memory chip of the wearable device, or passed to the gateway device (smartphone or smartwatch) through a wireless communication chip.

In various embodiments, the wearable device computes the pre-ejection period (PEP) by calculating the time-delay between the R-peak of the ECG, and the Aortic valve opening (AO) peak on the SCG signal. This is stored for every beat and/or averaged for a specified length of time (2 secs to 5 mins).

In various embodiments, the wearable device computes the PEP for the User during exercise for a specified time period (5 secs to 1 min) by storing the ensemble of all the beats (200-1200 ms from the R-peaks or 10-200 ms before the R-peak of each individual beat) and then looking for the AO peak in the ensemble signal.

In various embodiments, the wearable device computes the left-ventricular ejection time (LVET) by calculating the time-delay between the Aortic valve opening (AO) peak and the Aortic valve closure (AC) peak on the SCG signal. This is stored for every beat and/or averaged for a specified length of time (2 secs to 5 mins).

In various embodiments, the wearable device computes the LVET for the User during exercise for a specified time period (5 secs to 1 min) by storing the ensemble of all the beats (200-1200 ms from the R-peaks or T-peaks of each individual beat) and then looking for the AC peak in the ensemble signal.

According to various embodiments of the present invention, the Cardiac Time Intervals described herein are used to calculate a values for Stroke Volume (SV) and Cardiac Output (CO) in the form of:

SV=y₁*PEP+y₂*LVET+y₃*(PEP/LVET)+y₄*amp(AO)+y₅*IHR; or

SV=f(PEP,LVET,amp(AO),IHR); and

CO=SV*IHR;

Here, the constants yᵢ are typically regression coefficients derived from a training set consisting of a population database containing individuals in different age-groups, heights, weights, BMIs and prior medical histories, f is a linear or non-linear function. IHR denotes the instantaneous Heart Rate. In various embodiments, the regression coefficients, are determined separately for different age-groups or population groups with particular heights, weights and BMIs.

In various embodiments, the wearable device computes a value of the PEP gradient (ΔPEP) as:

ΔPEP=PEP(t₁)−PEP(t₂); or

ΔPEP=Avg(PEP(t₁ᵢ))−Avg(PEP(t₂ᵢ)); or

ΔPEP=Median(PEP(t₁ᵢ))−Median(PEP(t₂ᵢ)); or

ΔPEP=Slope(lsqfit(PEP(tᵢ))
Here PEP(t₁) and PEP(t₂) are the instantaneous PEP values on two consecutive beats, PEP(t₁ᵢ) and PEP(t₂ᵢ) are the PEP values in two consecutive intervals of time, each interval having a length of 1 sec-10 mins, the Avg is calculated after removing statistical outliers, and lsqfit(PEP(tᵢ) is the linear least-squares fit through the PEP values measured in a time interval tᵢ, of length 1 sec-10 mins, after outliers have been removed.

In various embodiments, the wearable device computes a value of cardiac fatigue using the value of ΔPEP described above, and measuring whether ΔPEP is positive for one or more time intervals (each time interval of length 1 sec-10 mins) during exercise. When such a condition is detected, the wearable device sends an alert to the User through the vibration motor and/or an audio speaker located on the wearable device and/or the gateway device. The system may further advise the User to hydrate and/or take rest and/or lower speed depending on the value of ΔPEP and the duration for which it was found to be positive.

In various embodiments, the wearable device computes a value of cardiac fatigue using the value of ΔPEP described above, and measuring whether ΔPEP is positive for one or more time intervals (each time interval of length 1 sec-10 mins) during exercise, when Heart Rate was either constant or increasing. When such a condition is detected, the wearable device sends an alert to the User through the vibration motor and/or an audio speaker located on the wearable device and/or the gateway device. The system may further advise the User to hydrate and/or take rest and/or lower speed depending on the value of ΔPEP and the duration for which it was found to be positive.

In various embodiments, the wearable device computes a value of cardiac fatigue using the value of ΔPEP described above, and measuring whether ΔPEP is positive for one or more time intervals (each time interval of length 1 sec-10 mins) during exercise, when Exercise Intensity (measured by Speed or standard deviation of the Y-axis accelerometer data) was either constant or increasing. When such a condition is detected, the wearable device sends an alert to the User through the vibration motor and/or an audio speaker located on the wearable device and/or the gateway device. The system may further advise the User to hydrate and/or take rest and/or lower speed depending on the value of ΔPEP and the duration for which it was found to be positive.

In various embodiments, the wearable device computes a value of the LVET gradient (ΔLVET) as:

ΔLVET=LVET(t₁)−LVET(t₂); or

ΔLVET=Avg(LVET(t₁ᵢ))−Avg(LVET(t₂ᵢ)); or

ΔLVET=Median(LVET(t₁ᵢ))−Median(LVET(t₂ᵢ)); or

ΔLVET=Slope(lsqfit(LVET(tᵢ))
Here LVET(t₁) and LVET(t₂) are the instantaneous LVET values on two consecutive beats, LVET(t₁ᵢ) and LVET(t₂ᵢ) are the LVET values in two consecutive intervals of time, each interval having a length of 1 sec-10 mins, the Avg is calculated after removing statistical outliers, and lsqfit(LVET(tᵢ) is the linear least-squares fit through the LVET values measured in a time interval tᵢ, of length 1 sec-10 mins, after outliers have been removed.

In various embodiments, the wearable device computes a value of cardiac fatigue using the value of ΔLVET described above, and measuring whether ΔLVET is negative for one or more time intervals (each time interval of length 1 sec-10 mins) during exercise, while PEP has remained the same or increased. When such a condition is detected, the wearable device sends an alert to the User through the vibration motor and/or an audio speaker located on the wearable device and/or the gateway device. The system may further advise the User to hydrate and/or take rest and/or lower speed depending on the value of ΔLVET and the duration for which it was found to be negative.

In various embodiments, the wearable device computes a value of cardiac fatigue using the value of ΔLVET described above, and measuring whether ΔLVET is negative for one or more time intervals (each time interval of length 1 sec-10 mins) during exercise, while Heart Rate has remained the same or decreased. When such a condition is detected, the wearable device sends an alert to the User through the vibration motor and/or an audio speaker located on the wearable device and/or the gateway device. The system may further advise the User to hydrate and/or take rest and/or lower speed depending on the value of ΔLVET and the duration for which it was found to be negative.

In various embodiments, the wearable device computes a value of the Cardiac Output (CO) gradient (ΔCO) as:

ΔCO=CO(t₁)−CO(t₂); or

ΔCO=Avg(CO(t₁ᵢ))−Avg(CO(t₂ᵢ)); or

ΔCO=Median(CO(t₁ᵢ))−Median(CO(t₂ᵢ)); or

ΔCO=Slope(lsqfit(CO(tᵢ))
Here CO(t₁) and CO(t₂) are the instantaneous CO values on two consecutive beats, CO(t₁ᵢ) and CO(t₂ᵢ) are the CO values in two consecutive intervals of time, each interval having a length of 1 sec-10 mins, the Avg is calculated after removing statistical outliers, and lsqfit(CO(tᵢ) is the linear least-squares fit through the CO values measured in a time interval tᵢ, of length 1 sec-10 mins, after outliers have been removed.

In various embodiments, the wearable device computes a value of cardiac fatigue using the value of ΔCO described above, and measuring whether ΔCO is negative for one or more time intervals (each time interval of length 1 sec-10 mins) during exercise, while Heart Rate and/or exercise intensity (measured through Speed or Variance in the Accelerometer Y-axis or Z-axis data) has remained the same or increased. When such a condition is detected, the wearable device sends an alert to the User through the vibration motor and/or an audio speaker located on the wearable device and/or the gateway device. The system may further advise the User to hydrate and/or take rest and/or lower speed depending on the value of ΔCO and the duration for which it was found to be negative.

In various embodiments, the wearable device is configured to send a real-time alert to the User if any cardiac fatigue and/or arrhythmia and/or abnormal ST-elevation and/or abnormal value of PEP/LVET/SV/CO are computed on the device. Such alerts are sent to the User by way of a vibration motor on the wearable device, and/or blinking of LEDs located on the wearable device, and/or a change on an electronic display on the wearable device, and/or an audio message issued through a speaker located on the wearable device.

In various embodiments, the wearable device is configured to send a real-time alert to the User if any cardiac fatigue and/or arrhythmia and/or abnormal ST-elevation and/or abnormal value of PEP/LVET/SV/CO are computed on the device. Such alerts are sent to the User by way of a message sent to the gateway device (smartphone or smartwatch) and communicated to the User by way of the vibration motor on the gateway device, and/or a notification displayed on the gateway device, and/or an audio message issued through the speaker located on the gateway device.

In various embodiments, the wearable device computes a value of Respiratory Rate (RR) in breaths per minute, by first calculating Respiratory cycles from the variation in QR-amplitudes measured from the ECG, and/or from a variation in the amplitude of the T-peak as measured in the ECG, and/or from the variation in the baseline of the ECG signal, obtained after applying a lowpass filter to the raw ECG signal with a cutoff at 1.5 Hz, and/or from the variation in the Z-axis of the accelerometer.

In various embodiments, the wearable device computes a value of Tidal Volume (TV) in ml or litres, by first calculating Respiratory cycles from the variation in QR-amplitudes measured from the ECG over a specified period of time, and then calculating the Tidal volume as:

TV=f(Max(QR_amp)−Min(QR_amp),Avg(QR_amp),Max(T_amp),Min(QR_amp))

Where QR_amp is the amplitude of R-peak of the ECG, measured in millivolts, and Tamp is the amplitude of the T-peak of the ECG, measured in millivolts.

In various embodiments, the wearable device computes a value of Minute Ventilation (VE) in liters per minute, using a combination of the Respiratory Rate (in breaths per minute) and Tidal Volume (in litres), to calculate Minute Ventilation as:

VE=RR×TV

In various embodiments, the health monitoring system contains an elastic strap, with a pressure sensor that measures the Respiratory cycles by recording the variations in the pressure felt in the pressure sensor on the body, and/or the variations in the tension of the strap measured by a spring embedded in the elastic strap. The strap sends the signals from the pressure sensor and/or the spring to the device coupled with the strap using a wired connection and/or a wireless communication module, which computes a value of the Respiratory Rate in breaths per minute.

In various embodiments of the present invention, when the User is being monitored while running, the wearable device computes the ground contact time (GCT, in seconds or milliseconds), and/or flight time (FT, in seconds or milliseconds) and/or cadence (Cd, in steps/min). When the accelerometer is worn in a manner such that it shows a value of 1 g while the User is standing, then the GCT is computed as the value above time between the zero-crossings when the value goes from negative to positive, and then back from positive to negative on the Y-axis of the accelerometer. The FT is calculated as the time between the zero-crossings when the value goes from positive to negative, and then back from negative to positive on the Y-axis of the accelerometer. These values are computed every 1-30 seconds, and stored on the memory chip on the wearable device, and/or sent to the gateway device via a wireless communication chip.

In various embodiments, the exercise health monitoring system uses the Heart Rate, PEP, LVET, Cadence, GCT, FT, Speed values from the User's data during a run, to compute the Optimal Cadence, GCT and FT value ranges corresponding to each particular speed that the User ran at, by calculating the cadence, GCT and FT values corresponding to each speed, where Heart Rate is minimal and/or PEP is maximum within the normal range, and/or LVET is maximum within the normal range, and/or the value of ‘Cardiac fatigue’ is the lowest. These values are communicated to the User to guide them towards the optimal running cadence and style.

In various embodiments, the Wearable device(s) worn on the torso on a chest strap, or clipped/stuck on some part of the leg computes the ‘shock’ (impact) on the knees and spine by calculating the maximum slope of the accelerometer's Y-axis (vertical direction) just before/after the foot strike on the ground. This shock is measured as the rate of change of deceleration (in g/sec) or as the deceleration itself, measured in g, when the accelerometer(s) are set to the +/−2 g or +/−4 g or +/−8 g range. The moment of the foot strike is computed as the 0 crossing on the Y-axis of the accelerometer, when the value goes from positive to negative, assuming that the orientation of the accelerometer(s) is such that it shows a value of 1 g in the standing position.

In various embodiments of the present invention, the ‘shock’ as described above, is computed in real-time on the MCU located on the wearable device, by looking for a pre-specified threshold in every set of new samples collected from the accelerometer(s) on the wearable device. If a pre-specified threshold (th) is crossed, then the maximum slope is computed from the previous s samples. Here s is the number of samples corresponding to 20-100 milliseconds, and th is a threshold between 2-8 g, depending on whether the accelerometer(s) are set to the +/−2 g or +/−4 g or +/−8 g range.

In various embodiments of the present invention, the exercise health monitoring system allows the User to configure a ‘shock limit’, so that he/she can be alerted in real-time as soon as the shock value as described above is computed, and if it crosses the particular shock limit that has been set. This alert is sent to the User as soon as a step that crosses the pre-specified shock limit is taken by way of a vibration and/or an audio message issued from the wearable device, so that they get real-time feedback on each foot strike, and can improve that running style or exercise regimen or stance while playing a sport.

In various embodiments of the present invention, the shock value, as described above, are computed for the left and right foot separately on one wearable device worn on the chest and/or two wearable devices worn on each foot. Once the values are computed separately for the left and right foot, the User gets real-time feedback through a vibration and/or changes in the LEDs and/or audio messages issued from the wearable device or the gateway device, on each foot strike, and a detailed report at the end of the exercise/run giving guidance on how to change the left or right foot strike, and differential forces being applied on each foot during exercise.

In various embodiments of the present invention, the wearable device computes a value of ‘Braking Force’, which is computed as the maximal deceleration along the Z-axis (forward facing direction) of the accelerometer situated on the wearable device worn on the chest or torso, right after each foot strike, and/or as the loss in velocity due to each foot strike. The foot strike is determined from the zero crossing on the Y-axis data as described above, and the braking force is calculated to be the minimum value on the Z-axis of the accelerometer in the 10-200 milliseconds following the foot strike.

In various embodiments of the present invention, the exercise health monitoring system allows the User to configure a ‘Braking limit’, so that he/she can be alerted in real-time as soon as the Braking value as described above is computed, and if it crosses the particular braking limit that has been set. This alert is sent to the User as soon as a step that crosses the pre-specified braking limit is taken by way of a vibration and/or an audio message issued from the wearable device, so that they get real-time feedback on each foot strike, and can improve that running style or exercise regimen or stance while playing a sport.

In various embodiments of the present invention, the braking value, as described above, is computed for the left and right foot separately on one wearable device worn on the chest and/or two wearable devices worn on each foot. Once the braking values are computed separately for the left and right foot, the User gets real-time feedback through a vibration and/or audio messages issued from the wearable device or the gateway device, on each foot strike, and a detailed report at the end of the exercise/run giving guidance on how to change the left or right foot strike, and differential forces being applied on each foot during exercise.

In various embodiments of the present invention, the wearable device computes a value of ‘Sway’, which is computed as the total deviation of the torso along the X-axis (left-right direction) in centimetres or in degrees from the vertical line on the path of running, during of each complete step. The duration of the step is taken to be the complete time from one foot strike till the next foot strike of the same foot, or the duration of two consecutive foot strikes.

In various embodiments of the present invention, the exercise health monitoring system allows the User to configure a ‘Sway limit’, so that he/she can be alerted in real-time as soon as the Sway value as described above is computed, and if it crosses the particular Sway limit that has been set. This alert is sent to the User as soon as a step that crosses the pre-specified Sway limit is taken by way of a vibration and/or an audio message issued from the wearable device, so that they get real-time feedback on each foot strike, and can improve that running style or exercise regimen or stance while playing a sport, to minimize Sway and thus increase efficiency during running/exercise.

In various embodiments of the present invention, the Sway value, as described above, is computed for the left and right foot separately on one wearable device worn on the chest. Once the Sway values are computed separately for the left and right foot (maximum deviation in centimeters or degrees in the left and right direction respectively), the User gets real-time feedback through a vibration and/or audio messages issued from the wearable device and/or the gateway device, on each foot strike, and a detailed report at the end of the exercise/run giving guidance on how to change the left or right foot strike, and differential forces being applied on each foot during exercise.

In various embodiments of the present invention, the computing device contained in the wearable device computes a value of ‘Bounce’ in centimetres, which is the total height by which the torso rises during one complete stride, while the the User is running.

According to various embodiments of the present invention, the computing device contained in the wearable device computes the speed of the User, while the User is running, using the following equation:

Speed=y₁*GCT+y₂*FT+y₃*(GCT)²+y₄*(FT)²+y₅*(GCT*FT); or

Speed=y₁*(1/GCT)+y₂*Cd+y₃*(1/GCT)²+y₄*(Cd)²+y₅*Height; or

Speed=f(GCT,FT,Cd,Max_{z},Max_{y},Int_{z},Int_{y}); and

Distance=Sumₜ(Speed(t)*time_interval(t));

Here, the constants yᵢ are typically regression coefficients derived from a training set consisting of a population database containing individuals in different age-groups, heights and weights, f is a linear or non-linear function, t is time, GCT and FT are measured in seconds, and Cadence (Cd) is derived as Cd=60*(1/(GCT+FT)) for each step. Max_{z} is the maximal acceleration in the Z-axis (forward direction) during the flight time, Maxₜ is the maximal acceleration in the Y-axis (upward direction) during the flight time, Int_{z} is the integral of the acceleration in the Z-axis (forward direction) during the flight time, Int_{y} is the maximal acceleration in the Y-axis (upward direction) during the flight time. In various embodiments, the speed is calculated for each step separately, or averaged for 1 sec-1 min. The distance is then calculated by summing the instantaneous products of the Speed and time over which the speed has been averaged. In various embodiments, the regression coefficients, are determined separately for different age-groups or population groups with particular heights and weights.

In various embodiments of the present invention, the readings from a gyroscope and/or magnetometer included in the device, are used to estimate the angle of the device with the ground that the User is running on, to correctly calculate the timepoint at which the User's foot touches the ground, and therefore to calculate a more accurate value of GCT and Flight time, and thereby a more accurate value of Speed, using the procedure described above.

In various embodiments of the present invention, the speed of a User while running is determined by first calculating the point in time t₀ at which the acceleration in the Y-axis crosses 1 g, or where the velocity in the Y-axis crosses 0, and taking another timepoint tᵢ, in the 10-100 ms range preceding t₀, where the velocity in the Z-axis at time tᵢ, V_{z}(tᵢ) is determined as:

V_{z}(tᵢ)=ₜ₀∫^{ti}a_{y}dt∫/(2*ₜ₀∫^{ti}a_{z}dt)+(ₜᵢ∫^{t0}a_{y}dt)/2

This procedure is repeated to calculate the velocity at multiple timepoints tᵢ, and therefore obtain a more accurate estimate of the velocity of the runner in the forward direction (V_{z}) at different time points. Once the velocity at any timepoint tᵢ is determined, the velocity at other timepoint tₛ in any continuous section of data is calculated by integrating the acceleration between the two timepoints as:

V_{z}(tₛ)=V_{z}(tᵢ)+ₜₛ∫^{ti}a_{z}dt

In various embodiments of the present invention, the readings from a gyroscope and/or magnetometer included in the device, are used to estimate the angle of the device with the ground that the User is running on, to correctly calculate the timepoint (t₀) at which the Users velocity along the vertical direction (V_{y}) is 0, and this is used to calculate the speed at any preceding point tᵢ, within 1-100 ms of t₀ as described above.

In various embodiments, the wearable device includes a GPS chip, capable of determining the exact position of the User during exercise or a run, and at the end of the exercise session/run, show how the various cardiac and musculo-skeletal parameters including but not limited to: HR, PEP, LVET, SV, CO, Shock, Cadence, Speed, Braking force, Sway: varied at different points of the route traversed by the User.

In various embodiments, the wearable device worn on the chest of the User includes a Barometer chip, which is capable of measuring the altitude of the User with respect to the sea level, and record changes in the altitude of the User with time.

In various embodiments, the exercise health monitoring system calculates a value for the Power with which the person is running (in Watts), using the measured values of GCT, Flight Time, Cadence, Maximal Acceleration in the Y and Z directions, rate of change of elevation (inclination), height and weight of the User. In other words, the Power (P) of the User is measured in Watts as follows:

P=f(GCT,FT,Cd,Max_{z},Max_{y},Inclination,Weight,Height)

In various embodiments, the wearable device computes a value of the Lactate Threshold (LT) and/or Ventilatory Threshold (VT) and/or Anaerobic Threshold (AT), by calculating the speed (in km/hr or m/sec) or pace (in seconds/km or seconds/mile) at which the Runner's Respiratory Rate suddenly starts increasing in a non-linear fashion with respect to pace and/or speed and/or Heart Rate and/or Power. VT is calculated to be the pace or speed at which the slope of the graph of RR vs Pace and/or RR vs HR and/or RR vs Power suddenly increases, after a linear increase over some period of time.

In various embodiments, the wearable device computes a value of the Lactate Threshold (LT) and/or Ventilatory Threshold (VT) and/or Anaerobic Threshold (AT), by calculating the speed (in km/hr or m/sec) or pace (in seconds/km or seconds/mile) at which the Runner's Minute Ventilation (VE) suddenly starts increasing in a non-linear fashion with respect to pace and/or speed and/or Heart Rate and/or Power. VT is calculated to be the pace or speed at which the slope of the graph of VE vs Pace and/or VE vs HR and/or VE vs Power suddenly increases, after a linear increase over some period of time.

In various embodiments, the exercise health monitoring system calculates values for RR, TV, VE, HR, Speed and Power for the runner in real-time, and guides a runner to run in their optimal zone, which is just below their Anaerobic Threshold or Ventilatory Threshold. The exercise health monitoring system does this by alerting the runner with a vibration and/or audio message, when they are approaching their Anaerobic Threshold or Ventilatory Threshold, and giving the runner a second longer vibration or different audio alert when they are in danger of crossing this AT/VT. Thus, until the runner receives the first alert, he/she knows they can exert themselves further, and if they receive the second alert, they know they need to slow down.

In various embodiments, the exercise health monitoring system, the wearable device and the App running on a smartphone/smartwatch together provide a test protocol, referred to herein as the ‘Ventilatory Threshold Test’ for the User. Under this protocol, the User is asked to wear the device, turn it on, and start walking on a Treadmill at a comfortable pre-specified pace. At fixed intervals of time, the User is alerted through a vibration and/or a sound to increase the speed of the Treadmill, and the User is asked to keep doing this until they receive an alert, or can go no further. Once this protocol is complete, the data recorded on the device, along with the durations and speeds the User walked/ran at, are used to compute the speed and/or Power at which the User reached their first ventilator threshold (VT1), their second Ventilatory Threshold (VT2), and their VO2-max level of exertion.

In various embodiments, the exercise health monitoring system consists of the wearable device worn on the body of the User, and a gateway device (smartphone or smartwatch) being carried by the User, which includes a GPS chip, capable of determining the exact position of the User during exercise or a run. This system is capable of showing, at the end of the exercise session/run, how the various cardiac and musculoskeletal parameters including but not limited to: HR, PEP, LVET, SV, CO, Shock, Cadence, Braking force, Sway: varied at different points of the route traversed by the User.

In various embodiments, the exercise health monitoring system consists of two wearable devices embedded in the soles of the left and right shoe of the User, each consisting of an accelerometer, and one or more of the following: a wireless communication chip, a battery, a computing device, a vibration motor, an energy harvesting circuit capable of harvesting energy from the pressure of the foot and charging the battery. These two wearable devices embedded in the shoes connect with a gateway device (smartphone or smartwatch), and compute one or more of the following parameters: cadence, speed, shock, braking, GCT, FT; and issuing alerts to the User by way of a vibration and/or messages sent to the gateway device during exercise/running. The wearable devices situated in the shoes are capable of recharging their batteries from the energy harvested from foot strikes.

In various embodiments, the exercise health monitoring system consists of one or more wearable devices worn on the chest or feet of the User, and a wireless headphone/earphone system worn by the User. This system is capable of measuring cardiac and musculoskeletal parameters including but not limited to: HR, PEP, LVET, SV, CO, Cardiac Fatigue, Shock, Cadence, Braking force, Sway; and sending real-time alerts to the User by way of vibrations and/or audio messages sent directly to the wireless headphones using a wireless communication protocol, without the use of any other gateway device. These alerts are played directly into the User's ears during exercise/running so that they can be alerted to different kinds of conditions.

In various embodiments, the User can wear one or more Wearable devices, which connect with a single gateway device such as a smart phone/smart watch/router wirelessly, using a wireless communication protocol such as Bluetooth or Wi-Fi. The Wearables first sync their internal clocks with the real time clock (RTC) of the gateway device—smartphone/smart watch, other gateway device—so that the internal clocks of the Wearables are aligned with the clock of the gateway device, as well as with each other. This synchronization is achieved by the smart phone sending its exact UTC time to the Wearables via Bluetooth, or some other wireless communication protocol, and the Wearables then updating their RTC to this exact time (to a resolution of milliseconds), plus a delta which has been calculated previously, and which is the time delay between the sending of the Bluetooth Low Energy (BLE) packet by the smart phone, and the updating of the RTC on the Wearable. This process of synchronization is optionally repeated every 1 hour or as needed, so that any drift between the clocks of different Wearables is normalized every 1 hour.

In various embodiments, the Wearable(s) transfer data to the smart phone/smart watch or other gateway device wirelessly via Bluetooth or some other near field communication (NFC) protocol, and then from there the data is transferred to a web client via Wi-Fi, and stored in a secure database on a web server. In another embodiment, the data is transferred directly from the Wearable to the web using Wi-Fi or 3G/4G wireless communication. This data can then be accessed by doctors or caregivers or the User themselves, using a web application, and historical data for each patient can be viewed and analysed.

In various embodiments, the data stored on the web for multiple Users, is used in Machine learning algorithms such as a convolutional neural networks and/or Bayesian Classifiers and/or support vector machines, to distinguish between healthy and pathological conditions of the User in question, by using the stored and annotated data as a training set, and applying the classification algorithms on the User's data in real-time on the MCU of the wearable device or the gateway device, while it is connected to the wearable device over Bluetooth.

In various embodiments, the system also allows the User or the caregiver to book appointments with a Physiotherapist/Doctor, and also to conduct an ‘Online Consultation’, by way of an audio/video VOIP call, or a regular phone call, or over a wireless communication protocol over the web, using the smart phone application that collects data from the Wearable. In this protocol, the User can also share his/her data with the Doctor or care-giver with whom the online consultation has been scheduled. During an online consultation, the system also allows the doctor or the concerned medical practitioner to view certain parts of the data generated by the wearable device, and to make episodic measurements from the Wearable device, through instructions delivered to the User, and thereafter to view the data in near real-time through a web application. In this manner, the system facilitates a ‘Virtual Examination’.

In various embodiments, the system stores the individual parameters, including but not limited to age, sex, medical history, for each user, and additionally combines anonymised information of multiple users from different locations on the cloud, in order to present each individual user with data about the health parameters of the user and activities in comparison with a similar population, and in order to give the user information about where they stand with respect to other users, in terms of percentiles and other comparative measures. Further, the system combines subjective information and information from multiple sensors on multiple users, as described above, to run Machine Learning algorithms, Bayesian classifiers or other kinds of training and testing protocols, in order to provide alerts that are customized for each individual, based on the normal parameters for the users in the same demographic section.

In various embodiments, the Wearable device records the ECG data at a frequency of anywhere between 125 Hz and 4 kHz, PPG data at a frequency of anywhere between 25 Hz and 2 kHz, Accelerometer data at a frequency of anywhere between 5 Hz and 2 kHz, and sends the data to the MCU, where the data is processed using mean/median/Band pass filters, and automated peak detection algorithms annotate each signal, and calculate the timing of the electrical, mechanical and blood-flow related events in the cardiac cycle.

In various embodiments, the pre-ejection period (PEP) is measured indirectly by calculating the time interval between the R-wave on the ECG and the J-wave, where the J-wave is assumed to be the highest maxima following the R-peak of the ECG, between 10-140 milliseconds after the R-peak of the ECG, in the y-axis data of the accelerometer, after removal of motion artifacts (R-Jᵢₙₜₑᵣᵥₐₗ). The equation used to derive PEP is of the form:

PEP=x₁*R−Jᵢₙₜₑᵣᵥₐₗ;
where the constant x₁ determined independently from his/her data, or from a population database.

According to another embodiment of the present invention, the pre-ejection period (PEP) is calculated before and during exercise being conducted by the User, and as the User is exercising, assuming that the User starts from rest at time t₁, the change in PEP due to exercise is calculated as: ΔPEP=PEPₜ₂−PEPₜ₁

Simultaneously, the power for any period of time Δt=t₂−t₁, is calculated as: P=F·S/Δt. The power (P), and the pre-ejection period (PEP) is calculated for every 10 sec interval, and the change in power (AP) and change in PEP (ΔPEP) is calculated between every two successive intervals. Then a value known as the Exercise PEP Index (EPEPI), is calculated as:

EPEPI=ΔP*ΔPEP.

According to other embodiments of the present invention, if the EPEPI index is calculated to be above a certain threshold thresh>0, then an alert is sent to the User through the vibration motor, or the smart phone application. This gives an indication to the User to not exert themselves any further, as the capacity of the heart to increase contractility further is exhausted. The exact value of thresh is calculated separately for each individual, on the basis of his/her age, fitness levels, prior medical history.

In various embodiments, the Wearable device records the ECG data as described above, and uses an algorithm running on the MCU, which uses the 3-10 data points around the P/Q/R/S/T peak, and then uses wavelet transforms or other peak-interpolation techniques to further improve the accuracy of the detected peaks to less than 1 millisecond.

According to various embodiments, the Wearable device uses data from the ECG sensor, PPG sensor, and the SCG as measured from the accelerometer, as described herein, and when the Wearable device is affixed on some part of the chest, to calculate values for the cardiac time intervals (CTIs) including, but not limited to: Pre-ejection period (PEP), left ventricular ejection time (LVET), Q-wave of ECG to the first sound from the Phonocardiogram (QS1), Q-wave of ECG to the second sound from the Phonocardiogram (QS2), first sound to second sound of Phonocardiogram (S1S2), PR-interval from ECG, QRS duration from ECG, the time interval between the R-wave on the ECG, the J-wave on the y-axis of the SCG (R-J interval), Systolic Time, Diastolic Time, PTT_{foot}, PTTₚₑₐₖ, Electro-mechanical Activation time (R-peak to MC), Isovolumetric Relaxation Time (IVRT), which is the time interval from AC to MO on the Seismocardiogram, and Isovolumetric Contraction Time (IVCT), which is the time interval from MC to AO, and/or the like.

In various embodiments of the system, the values of PEP and LVET as calculated from the Wearable device, as described elsewhere herein, are used to calculate a beat-to-beat value of PEP, LVET and PEP/LVET, which is combined with values of instantaneous heart rate (IHR), as measured by ECG, Pulse Wave Velocity (PWV), as measured from ECG and PPG, amplitude of PPG peak, amplitude of AO peak, body mass index (BMI), to arrive at a measure of a Cardiac Health Index (CHI). In other words, we derive a function:

CHI=x₁*PEP+x₂*LVET+x₃*(PEP/LVET)+x₄*amp(AO)+x₅*amp(PPG)+x₆*IHR+x₇*PWV; or

CHI=J(PEP,LVET,R−Jᵢₙₜₑᵣᵥₐₗ,amp(AO),amp(J-wave),amp(PPG),IHR,PWV,SpO₂,height,weight,BMI,age,blood profile,genetic profile);

Here, Where the constants xᵢ are calculated as a weighted average of the User's age, Body Mass Index (BMI), height, weight and other medical history and Blood lipid profile (if available) and gender, and f is a linear or non-linear function that maps the input parameters to the output variable CHI.

In other embodiments of the invention, the values mentioned in [39] above are used to calculate the Tei index, or the Myocardical Performance Index, as MPI=(IVRT+IVCT)/LVET. This value is calculated for some or every heartbeat on the Wearable device, and is sent to the accompanying smart phone or other gateway device via Bluetooth, or some other NFC protocol.

In the present invention, the signal quality of each individual sensor is optionally assessed and a confidence interval is calculated for signals including ECG, PPG and SCG. In order to assess signal quality, the method of (a) template matching, after normalizing the y-axis values between min and max (i.e. between 0 and 1), and by normalizing the x-axis in such a manner so that at least 2 peaks (R-peaks from ECG, AO-peaks from SCG, Pulse wave peak from PPG) in the window occur at the same positions; (b) comparison of FFTs of the measured and ideal signals; (c) comparison of the Histograms of the measured and ideal signals.

According to some embodiments of the present invention, the system for health monitoring is configured in such a way that the ECG and SCG measured on the chest with a Wearable are combined with PPG signals measured on a User's fingers from the LED sensor on a smartphone, and the internal clock of the Wearable is synced with the internal clock of the smart phone, and the signals from the Wearable to an application running on the smart phone, which combines the data from the Wearable and the internal PPG sensor on the smart phone, to arrive at values for PWV_{foot} and PWVₚₑₐₖ, which are used to estimate values for peripheral SBP and DBP, as well as other parameters of cardiovascular health, which depend upon the PWV, such as LVEF, or risk of Ischemia and atherosclerosis.

According to some embodiments of the present invention, the system for health monitoring is configured in such a way that the ECG and SCG measured on the chest with a Wearable are combined with PPG signals measured on a User's wrist from a smart watch, and the internal clock of the Wearable is synced with the internal clock of the smart watch, and the signals from the Wearable to the smart watch, which combines the data from the Wearable and the internal PPG sensor, to arrive at values for PWV_{foot} and PWVₚₑₐₖ, which are used to estimate values for SBP and DBP, as well as other parameters of cardiovascular health, which depend upon the PWV.

### DETAILED DESCRIPTION OF THE FIGURES

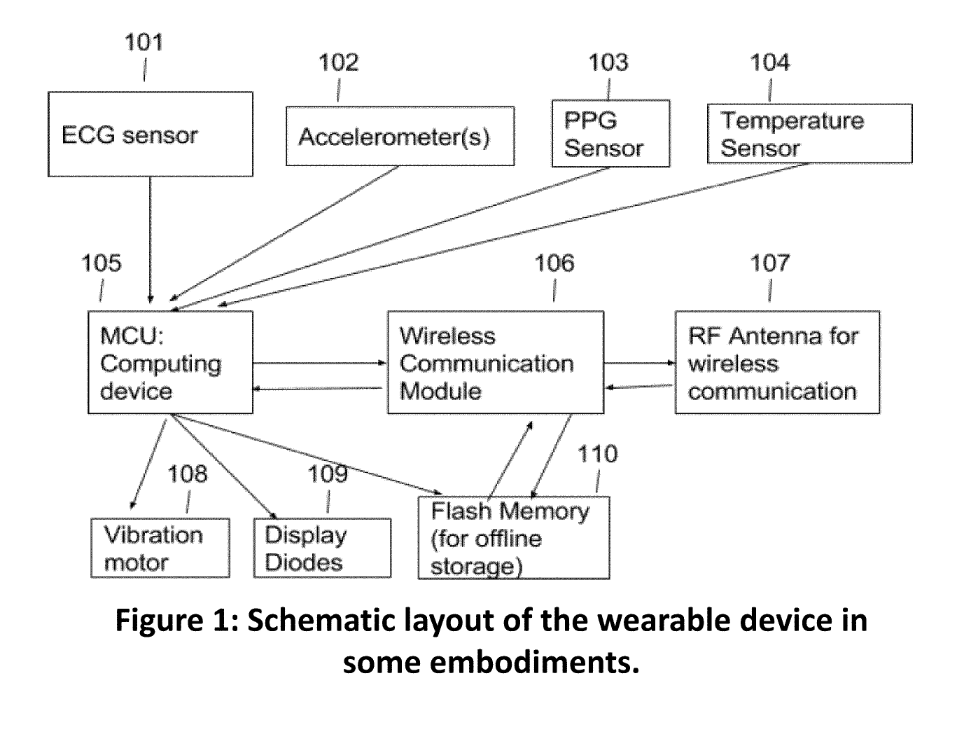

FIG. 1 shows a schematic layout of the Wearable Device (115) and shows a plurality of sensors including an electrical sensor (101), accelerometer(s) (102), PPG sensor (103), Temperature sensor (104) that record data, and send the data to a microcontroller or microprocessor or another computing device (105) on the Wearable.

As shown in FIG. 1, the computing device (105) receives outputs from the sensors (101, 102, 103, 104). The computing device (105) is configured to process this data as described elsewhere herein, and then send the data and/or results of the processing for transmission to the BLE chip (106). The wireless transmission module or BLE chip (106) then sends this data to the BLE antenna (107), which then communicates the data to other devices.

FIG. 1 also shows that in certain instances, on the Wearable device includes a memory chip (110). Memory chip (110) is configured to store data received from the sensors (101, 102, 103, 104), and/or the output of microprocessor (105). The stored data may later be transmitted to another device. The Wearable device also includes status diodes (109) which indicate different states of the device, and a vibration motor (108), which can alert the user when configured to do so, either by the MCU (110) or upon receiving an instruction from the smart phone/smart watch (116).

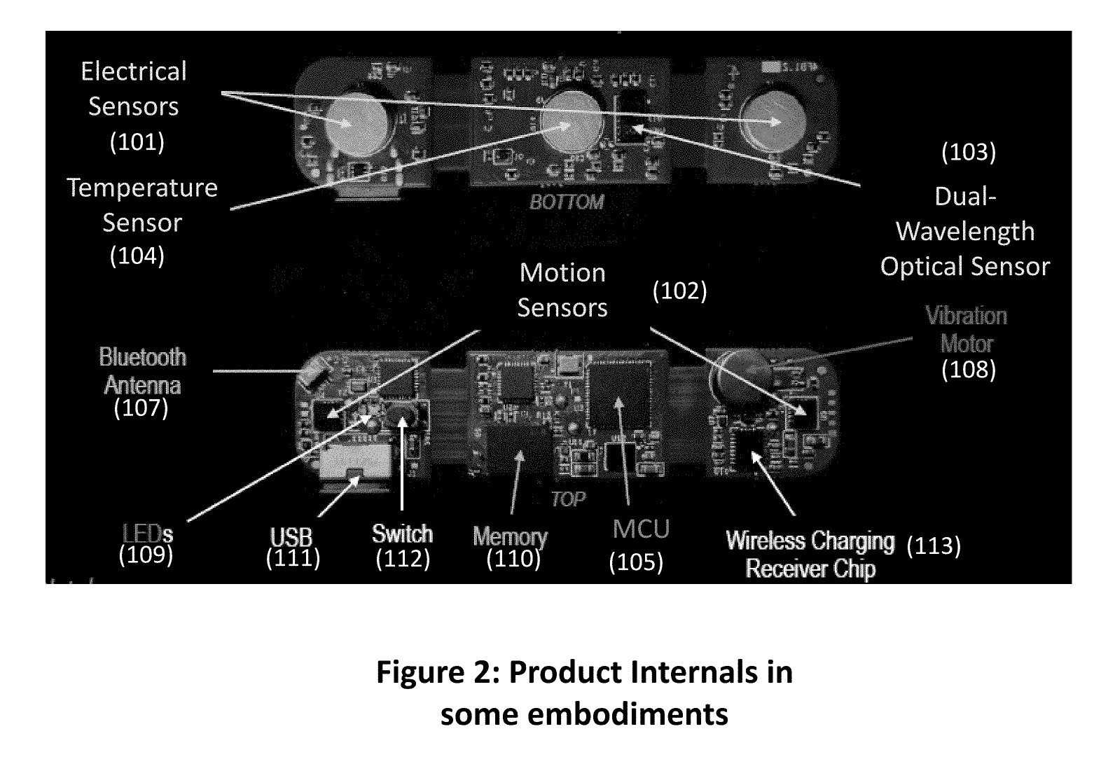

FIG. 2 shows a picture of the internal components of the Wearable, in the actual configuration, as used in the product, according to various embodiments. The picture on the top shows the lower side of the PCBs, comprising the electrical sensors (101), the optical sensors (102), and the temperature sensor (104). The picture on the bottom shows the upper side of the PCBs, comprising of the accelerometers (102), the LEDs (109), the Bluetooth antenna (107), the USB port (111), the switch (112), the memory chip (110), the processor (105), the vibration motor (108) and the wireless charging receiver chip (113).

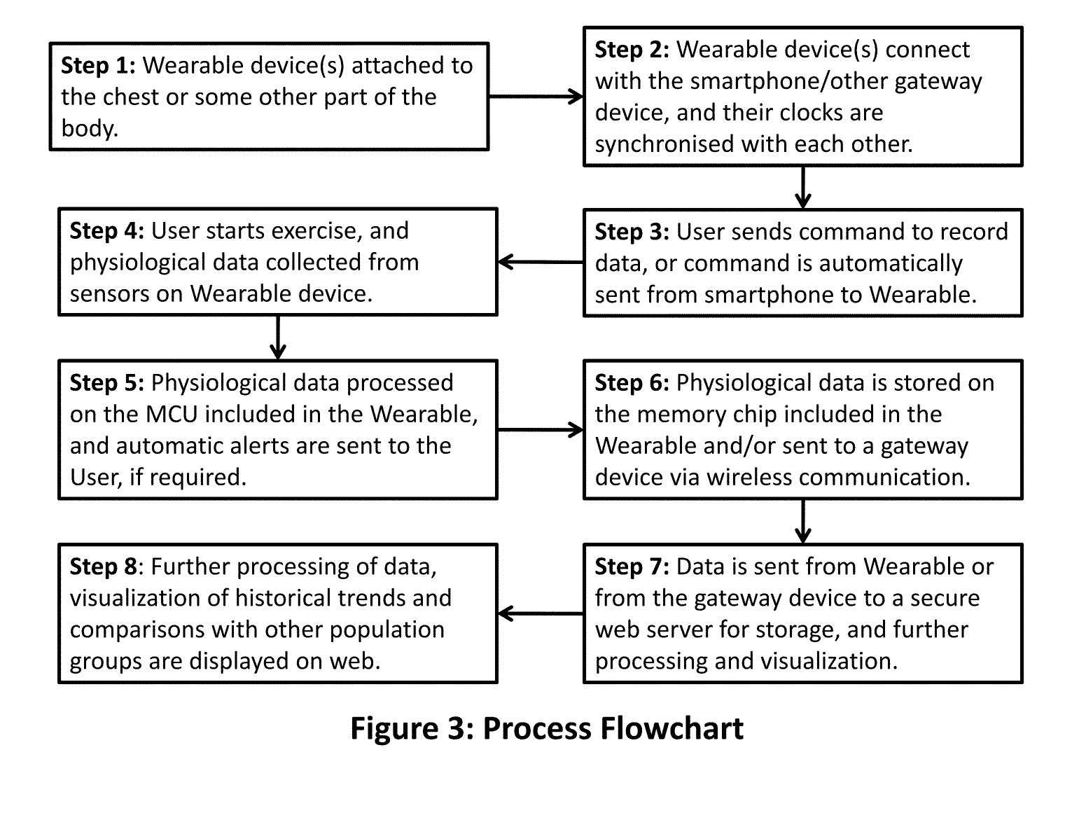

FIG. 3 shows a process flowchart for how the Wearable and accompanying application function, in various embodiments. In Step 1, one or more Wearable devices are attached to some parts of the User's body. In Step 2, the Wearable devices connect with the smartphone or gateway device. In Step 3, the User or the User sends a command from the smart phone to the Wearable devices, to record physiological data from the User's body. In Step 4, physiological data is recorded by the Wearables from sensors on the device. In Step 5, the data collected is processed by the MCU on the Wearable device. In Step 6, this processed data is stored on a memory chip on the device, or sent to the smart phone using some wireless communication protocol. In Step 7, this data is also sent from the Wearable or smart phone to a secure storage location on the Cloud. In Step 8, the data is optionally further processed, and other insights are derived by processes running on the web server, as described elsewhere herein.

FIG. 4 shows the process flowchart for the Arrhythmia detection algorithm which runs on the wearable device. In Step 1, the AFE sensor on the wearable device records the single-lead ECG signal on the User's chest. In Step 2, the raw ECG signal is stored on the memory chip located within the device. In Step 3, an algorithm running on the device denoises the signal. In Step 4, sections of the denoised ECG signal are passed to a CNN Classification object. In Step 5, the ECG sections are classified as “Normal” or “Arrhythmic” based on the output of the algorithm. In Step 6, an automatic alert is issued in the case of Arrhythmia detection. In Step 7, the ECG data is sent to a gateway device, and from there to the web for secure storage. In Step 8, the data is further processed and visualized on the web.

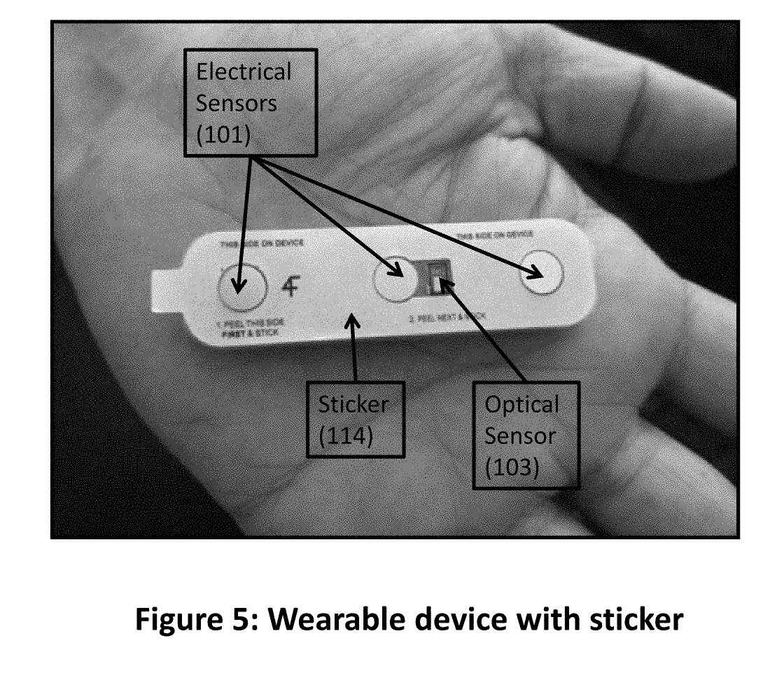

FIG. 5 shows one embodiment of the Wearable device (115), which can be affixed to a User's body with a double-sided sticker (114), which contains cut-out holes for the Electrical Sensors (101) and the Optical Sensor (103), so that they can be in direct contact with the User's skin.

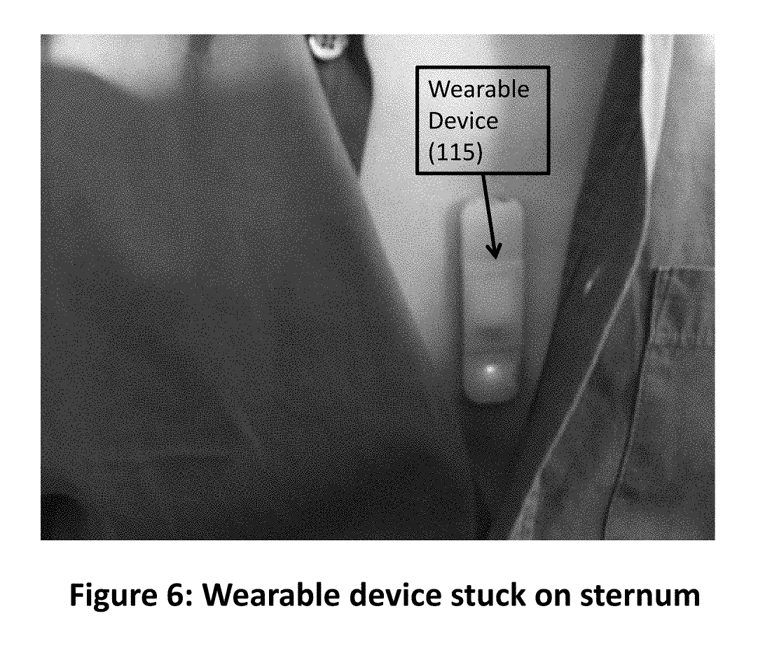

FIG. 6 shows one embodiment of the Wearable device (115), affixed to a User's sternum with the help of a double-sided sticker.

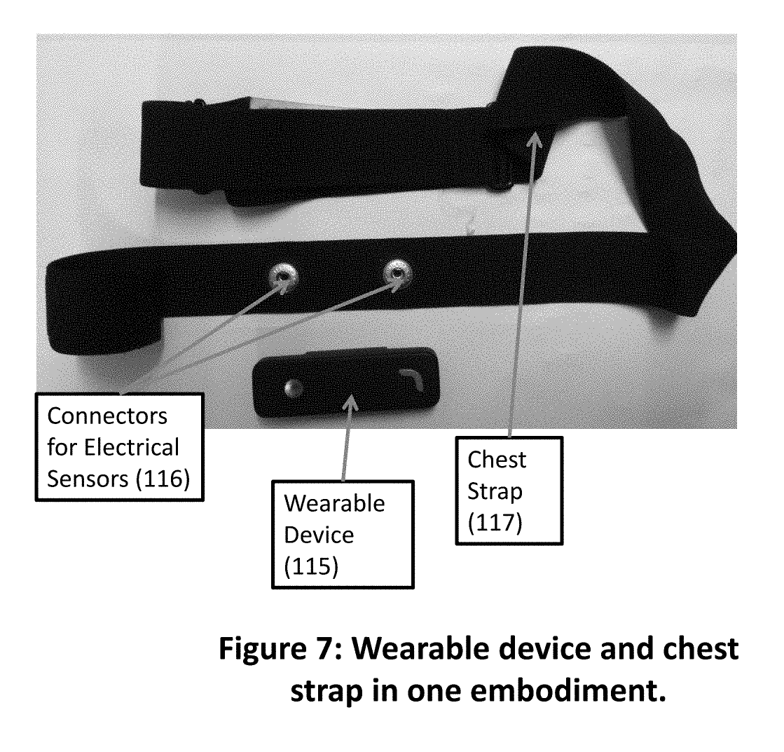

FIG. 7 shows one embodiment of the Wearable device (115), which can be affixed to a User's body with a chest strap (117), which contains metallic button connectors (118) where the Wearable device can be clipped on, and through which the electrical signals are transmitted.

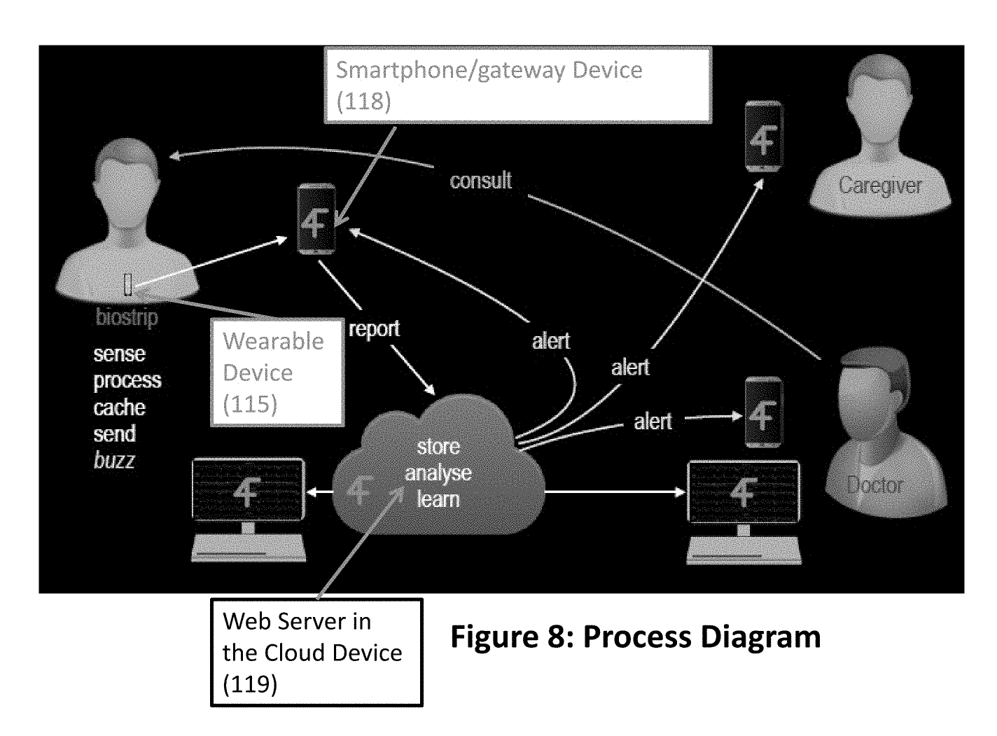

FIG. 8. shows one embodiment of the Health Monitoring system, where the wearable device (115) is attached to the User's chest, and connects with a smartphone or gateway device (118). The physiological data collected by the device is first transmitted to the smartphone or wearable device (118), and from there to the Cloud server (119).

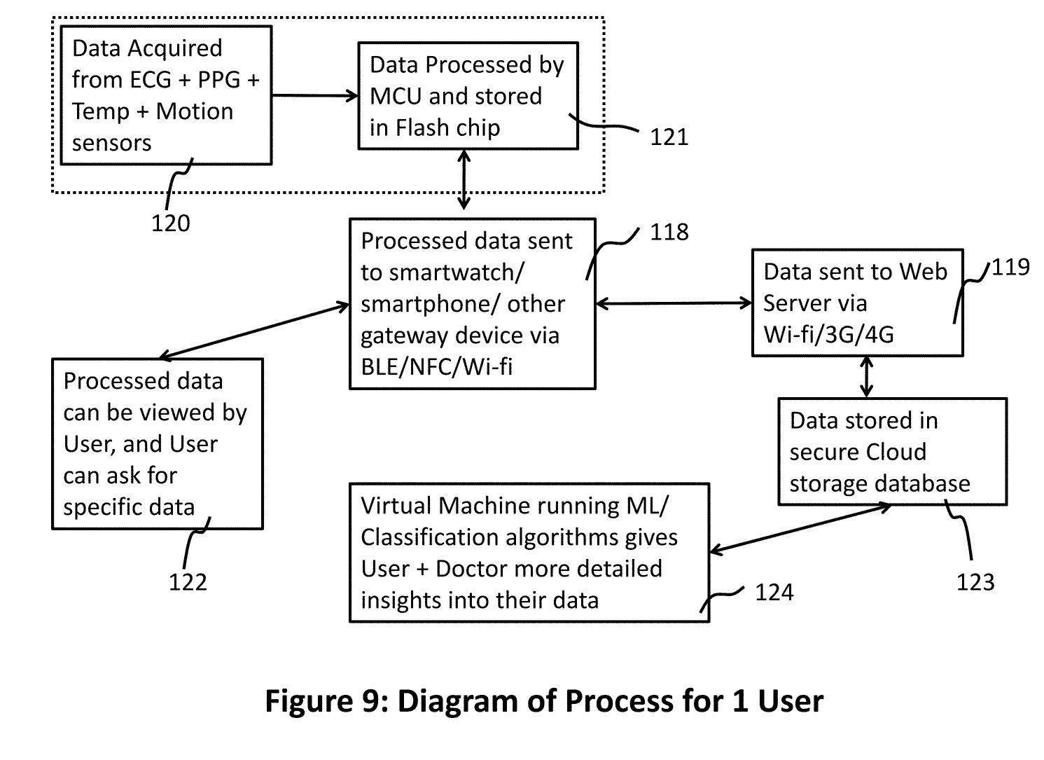

FIG. 9 illustrates various embodiments of the process of monitoring a single patient. In this embodiment, the system collects (120) the data from one or more Wearable devices (115), and then processes (121) the data on the MCU on board the Wearable device (115). The data, after it is processed by the MCU, is sent to the smart phone/smart watch (118) or other gateway device via a wireless communication protocol. From the smart phone/smart watch (118), the data can be sent (122) to the User, or it can be sent to the web server (119) via Wi-Fi or some other wireless communication protocol. From the web server, the data can be sent to a secure Cloud database (123) for storage. The data stored in the Cloud database is then accessed (124) by Machine Learning algorithms that can access this data and give the User or a Doctor more detailed insights into the data, and also push these insights into the Cloud database.

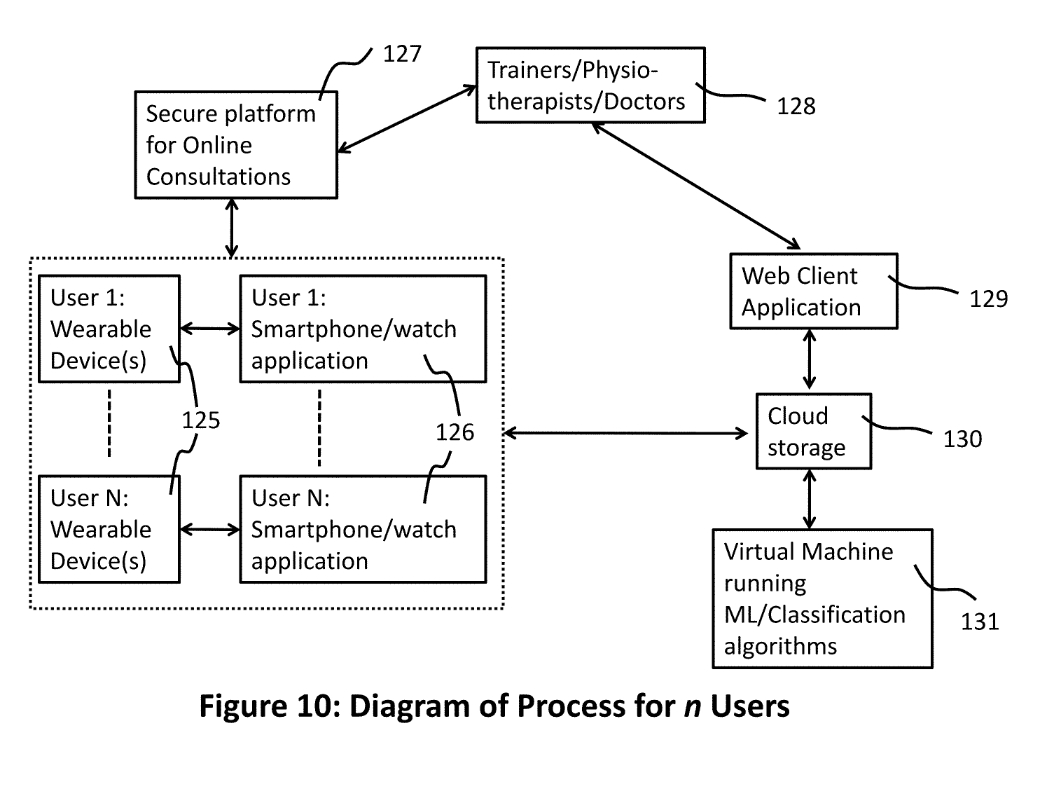

FIG. 10 shows a block diagram for the collection of physiological data by multiple Wearables for multiple Users (125), in various embodiments. The data collected (125) from multiple Users and Wearables, is sent (126) to the smart phones of the respective Users. From the smart phones, the data can be sent (127) to a secure platform for online consultation, which can then be accessed (128) by a Doctor/Caregiver, or be sent (129) to a web client application, or be sent (130) to the Cloud for storage purposes. From the Cloud storage (130), the data can be accessed (129) through a web client application. The data can also be accessed (131) by Machine Learning algorithms that can derive insights from this data.

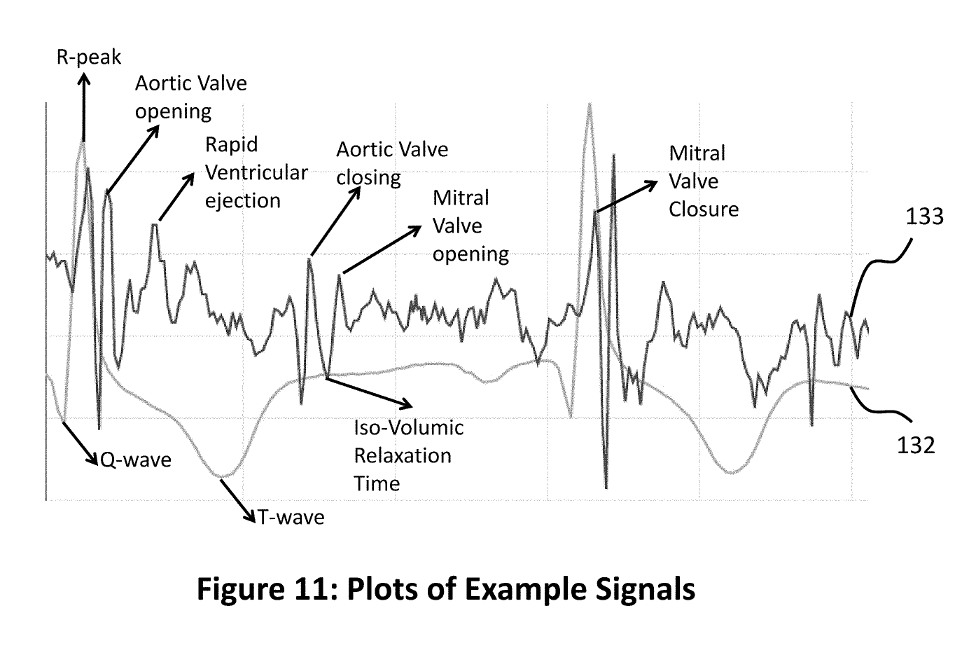

FIG. 11 shows an example of the signals captured on the Wearable device (115), comprising of the ECG signal (132) and the SCG signal (133), which together denote several events of the cardiac cycle, including the P, Q, R, S and T complexes from the ECG, and the Aortic valve opening (AO), Aortic valve closure (AC), Mitral valve opening (MO), Mitral valve closing (MC), Rapid Filling (RF) and Rapid Ejection (RE) events from the Seismocardiogram. These are together used to calculate the cardiac time intervals discussed herein.

### Advantages of the Invention

The present invention enables the continuous monitoring or various health parameters, particularly cardiac health parameters, over long periods of time due to the use of a wearable device that can be affixed to the body for long periods of time.

The present invention enables monitoring of health parameters in a fully mobile setting, hence allowing subjects to be monitored during exercise or a run covering large distances.

The present invention includes a wearable device that is capable of processing data streams being received from various sensors, and is further capable of computing derived health parameters on the device itself.

The present invention allows the User to be alerted discreetly using a vibration motor and/or an audio speaker whenever the calculated health parameters are found to be outside the normal range of values to be expected from that parameter.

The present invention allows the Users health parameters to be sent to an application running on the smart phone/smart watch or other gateway device, and also allows the data to be stored in a secure web location, so that the data can be viewed and analysed by health professionals at a later stage.

The foregoing description of the specific embodiments will so fully reveal the general nature of the embodiments herein that others can, by applying current knowledge, readily modify and/or adapt for various applications such as specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modifications. However, all such modifications are deemed to be within the scope of the claims.

What is claimed is:

1. A wearable device capable of being affixed onto the body of the User during exercise, comprising:
a) one or more PCBs;
b) a number of physiological sensors, including but not limited to at least two of: ECG sensor and Skin Impedance sensor and PPG sensor, Accelerometers and temperature sensor;
c) a computing device configured to record data from any subset of the sensors;
d) a wireless communication chip capable of sending data to a gateway device such as a smartphone or smartwatch or router.

2. The device of claim 1, further including a vibration motor and/or LEDs and/or an audio speaker, capable of sending real-time alerts to the User during exercise or resting states.

3. The device of claim 1 further including a chest strap or a disposable sticker that allows the device to be affixed to some part of the User's chest, wherein the physiological sensors are configured to record signals including but not limited to: ECG and Accelerometer waveforms.

4. The device of claim 1 wherein one of the PCBs further includes one or more of the following:
a USB port;
a gyroscope;
a magnetometer;
a barometer;
a battery;
a temperature sensor;
a GPS chip;
one or more LEDs;
a thermoelectric or photoelectric panel for harvesting energy from the body heat, or from light or heat in the environment;
an Ultrasound transducer, configured for recording an Ultrasound signal;
an electronic display; and
a wireless charging coil.

5. A device, according to claim 1, wherein the computing device included in the wearable is further configured to measure ECG and SCG waveforms in parallel, when affixed to the chest of the User with a strap or adhesive sticker, and further configured to derive parameters related to the cardiovascular health of the individual, including one or more of the following: Heart Rate, Respiratory Rate, Tidal Volume, Minute Ventilation, Arrhythmias, Heart murmurs, Systolic Time Intervals (PEP, LVET, IVRT, IVCT), Left Ventricular Ejection Fraction (LVEF), Cardiac Output and Stroke volume, PQ-interval, ST-interval, ST-elevation, Running speed, Running power.

6. A device according to claim 1, wherein the computing device is configured to record the accelerometer data, and to calculate the exact timing of the zero crossings in the Y-axis data of the accelerometer, and thereby a value for one or more of the following when the User is undergoing exercise, or is running: cadence (in steps per min), shock on the knees/spine (in g/sec), braking force (in g), braking velocity (in m/sec or km/hr), bounce (in cms), sway (in cms or degrees from the vertical), ground contact time (in sec/ms), flight time (in sec/ms), speed (in m/sec or km/hr).

7. The device, according to claim 1, wherein the computing device is configured to measure the ‘shock’ value by computing the maximal slope of the Y-axis data of the accelerometer in the 1-100 ms time interval range immediately after the foot strike, and send an immediate alert to the User when their shock value crosses a certain pre-specified threshold, through a vibration motor, and/or a message displayed on a display included in the device, and/or an audio speaker located on the wearable device, and/or a message sent to a gateway device such as a smartphone or smartwatch.

8. The device, according to claim 1, wherein the computing device is configured to measure the ‘braking’ value by computing the minimal value of the Z-axis data of the accelerometer 1-100 ms immediately after the foot strike, or as a loss of velocity (in m/s or km/hr) due to each footstrike, and send an immediate alert (within 1 ms-10 sec) to the User when their braking value crosses a certain pre-specified threshold, through a vibration motor, and/or a message displayed on a display included in the device, and/or an audio speaker located on the wearable device, and/or a message sent to a gateway device such as a smartphone or smartwatch.

9. The system according to claim 1, further configured to compute a value for the ‘Ventilatory Threshold’ or ‘Lactate Threshold’ or ‘Anaerobic Threshold’ for runners, by estimating the speed at which the runner's Respiratory Rate or Minute Ventilation goes beyond a pre-specified threshold, or starts increasing non-linearly with respect to speed and/or Heart Rate and/or Power for a certain period of time. The system is further capable of alerting the User through a vibration, and/or a message displayed on a display included in the device, and/or audio message whenever the User is close to this threshold, thereby enabling the User to run in a zone close to their Anaerobic Threshold.

10. A system for monitoring the cardiovascular health of any mammal, comprising:
a) One or more wearable devices, each including two or more of the following physiological sensors: ECG sensor, PPG sensor, Accelerometers; and a wireless communication module.
b) A gateway device, such as a smartphone or smartwatch or router, configured to obtain signals from the wearable device;

11. The system, according to claim 10, wherein the device is affixed to some part of the User's chest using a strap or a disposable sticker while the User is exercising, and the wearable device is configured to measure the values of one or more of the following: PEP, LVET, HR, Respiratory Rate, Tidal Volume, Cadence, Braking Force, Braking Velocity, Bounce, Shock, Sway.

12. The system, according to claim 10, wherein the exercise health monitoring system consists of the wearable device worn on the body of the User, and a gateway device (smartphone or smartwatch) being carried by the User, which includes a GPS chip, capable of determining the exact position of the User during exercise or a run. This system is capable of showing, at the end of the exercise session/run, how the various cardiac and musculoskeletal parameters including but not limited to: HR, PEP, LVET, SV, CO, Shock, Cadence, Braking force, Sway: varied at different points of the route traversed by the User on a display included in the device, and/or on the smartphone or other gateway device.

13. The system, according to claim 10, further configured to compute a value of ‘Cardiac Fatigue’ using the value of PEP/SV/LVET/CO gradient calculated by taking the slope of two or more consecutive (beat-to-beat) values, and measuring whether the PEP gradient is positive or LVET/SV/CO gradient is negative for one or more time intervals (each time interval of length 1 sec-10 mins) during exercise, when Exercise Intensity (measured by Heart Rate or Speed or standard deviation of the Z/Y-axis accelerometer data) was either constant or increasing.

14. The system according to claim 10, further configured to compute a value for the ‘Ventilatory Threshold’ or ‘Lactate Threshold’ or ‘Anaerobic Threshold’ for runners, by estimating the speed at which the runner's Respiratory Rate or Minute Ventilation starts increasing non-linearly with respect to speed and/or Heart Rate and/or Power for a certain period of time. The system is further capable of alerting the User through a vibration, and/or a message displayed on a display included in the device, and/or audio message whenever the User is close to this threshold, thereby enabling the User to run in a zone close to their Anaerobic Threshold.

15. The system according to claim 10, further configured to issue alerts through a vibration motor and/or an audio speaker located on the wearable device and/or the gateway device when a condition of Cardiac Fatigue or Ventilatory Threshold is detected. The system may further advise the User to hydrate and/or take rest and/or lower speed.

16. The system according to claim 10, wherein the internal clocks of the wearable device and a smartwatch are synchronized, and the time delay between the R-peak of the ECG or the Aortic valve opening on the SCG, as measured on the wearable device, and the peak of the PPG pulse, as measured on the smartwatch, is calculated on the smartwatch to obtain a value of the Pulse Transit Time, and further use this value to estimate the Blood Pressure of the User.

17. A system for monitoring the health of any human being in a mobile setting, comprising:
a) One or more PCBs containing one or more of the following physiological sensors: ECG sensor, PPG sensor, Accelerometers; and a computing device and a wireless communication module.
b) A chest strap or shirt or vest capable of housing the above-mentioned PCBs, and keeping them affixed to some part of the User's chest;
Capable of recording the ECG of the User, and measuring one or more of the following parameters: PEP, LVET, HR, Respiratory Rate, Tidal Volume, Cadence, Braking Force, Braking Velocity, Bounce, Shock, Sway.

18. A system, according to claim 17, wherein the PCB or chest strap or vest further contains one or more of the following:
a port for charging with a wire;
a gyroscope;
a magnetometer;
a barometer;
a battery;
a temperature sensor;
a GPS chip;
a pressure sensor to measure the pressure between the strap an the User's chest;
a strain sensor to measure the strain in the chest strap or vest when it is worn by the User;
one or more LEDs;
a thermoelectric or photoelectric panel for harvesting energy from the body heat, or from motion, or from light/heat in the environment;
an Ultrasound transducer, configured for recording an Ultrasound signal;
an electronic display;
a wireless charging coil.

19. A system, according to claim 17, where the wireless communication chip sends data to a gateway device such as a smartphone or smartwatch, which displays the parameters calculated by the computing device contained within the device, and which can be used to configure the wearable device for different functions.

20. A system according to claim 17, wherein the strap contains an array of electrical sensors capable of recording multi-channel ECG and/or impedance, and further capable of measuring one or more of the following: Respiratory Rate, Tidal volume, VO2 max, fluid content in the lungs, ST-elevation, Left Ventricular Ejection Fraction, Stroke Volume, Cardiac Output, Galvanic Skin Response.

21. The system according to claim 17, further configured to compute a value for the ‘Ventilatory Threshold’ or ‘Lactate Threshold’ for a User during exercise, by estimating the exercise intensity level at which the User's Respiratory Rate or Minute Ventilation starts increasing non-linearly with respect to exercise intensity level and/or Heart Rate and/or Power for a certain period of time. The system is further capable of alerting the User through a vibration, and/or a message displayed on an electronic display included in the device, and/or audio message whenever the User is close to this threshold, thereby enabling the User to run in a zone close to their Anaerobic Threshold.
